(19) United States
(12) Patent Application Publication
(10) Pub. No.: US 2020/0245850 A1
(43) Pub. Date: Aug. 6, 2020

(54) ILLUMINATING SHEATH AND SHEATH POSITION SENSING SYSTEM

(71) Applicant: Spiration, Inc. d/b/a Olympus Respiratory America, Redmond, WA (US)

(72) Inventors: Taylor N. Tyson, Seattle, WA (US); Anthony H. Siuda, Woodinville, WA (US); Michael S. Smith, Sammamish, WA (US)

(21) Appl. No.: 16/263,985

(22) Filed: Jan. 31, 2019

(51) Int. Cl. A61B 1/00; A61B 1/267; A61B 1/018; C09K 11/00; A61B 1/05; A61B 8/00; A61B 1/005

(52) U.S. Cl. CPC A61B 1/00135; A61B 1/2676; A61B 1/018; A61B 1/005; A61B 1/05; A61B 8/4483; C09K 11/00

(57) ABSTRACT

A self-illuminating, flexible sheath includes a distal end, a proximal end, and a lumen extending from the proximal end to the distal end. The flexible sheath includes a self-luminating feature. The flexible sheath is configured to be received by a working channel of an endoscope.

### BACKGROUND

Sheaths on medical devices are typically used to protect bronchoscopes and endoscope working channels in addition to supporting the needle during penetrations.

Existing devices that are used for bronchoscopy do not include means of preventing health care professionals (HCPs) from overextending the sheath and potentially damaging airway walls or other tissue. Most devices have a set length sheath allowing a user to bring the sheath into the view of the endoscopic camera and closer to the target tissue. This set length is adjusted from the handle, but there are no feedback mechanisms to prevent the HCP from overextending the sheath into the tissue. To avoid overextension, some HCPs will set the sheath extension length while the scope is outside of the body. They extend the sheath to the maximum length they feel comfortable with and then lock the sheath at that position. This takes extra time, though, and requires the HCPs to remove the scope from the body, if not done before beginning the procedure.

### SUMMARY

The present disclosure relates to an improvement in the way health care professionals (HCPs) confirm the sheath and device has exited the working channel of the instrument and provides another type of visual indication that it is safe to deploy the needle without fear of damaging a bronchoscope or tissue in the process of an operation.

The present disclosure provides an exemplary flexible sheath that includes a distal end, a proximal end, and a lumen extending from the proximal end to the distal end. The flexible sheath includes a self-luminating feature. The flexible sheath is configured to be received by a working channel of an endoscope. The endoscope may be a bronchoscope.

In one aspect, the self-luminating feature includes a luminous pigment that is applied as a paint to at least a portion of the flexible sheath or is added during manufacturing of the flexible sheath.

In another aspect, the flexible sheath includes a first chamber section, a second chamber section, and a breakable membrane located between the first and second chamber sections. The self-luminating feature includes at least a first solution located in the first chamber section and at least a second solution located in the second chamber section. When the first solution comes into contact with the second solution, a chemiluminescence activity occurs.

In still another aspect, a system includes an endoscope system and a flexible sheath. The endoscope includes a handle, an insertion tube and an imaging system. The insertion tube includes a proximal end coupled to the handle, a distal end, a working channel, and an exit port of the working channel at the distal end. The imaging system includes an ultrasound transducer located at the distal end of the insertion tube, an imaging sensor configured to produce image data, an image processor configured to receive the produced image data from the image sensor and generate an output signal based on the received image data, and an output device configured to output the generated output signal. The imaging sensor includes a component located proximal of the ultrasound transducer and distal of the exit port at the distal end of the insertion tube. The flexible sheath is slidably received within the working channel. The flexible sheath includes a distal end, a proximal end, and a lumen extending from the proximal end to the distal end. The flexible sheath includes a self-luminating feature.

In yet another aspect, the image processor is configured to analyze the image data received from the image sensor and generate an indication about the distal end of the flexible sheath based on the analyzed image data. The output device outputs the generated indication. The image processor analyzes at least one of an intensity value or a wavelength value and generates the indication if the analyzed intensity value or the wavelength value is greater than a predefined threshold.

In still yet another aspect, the image processor analyzes the image data received from the image sensor and generates a value of a position of the distal end of the flexible sheath relative to the exit port based on the analyzed image data. The output device outputs the generated value. The image processor analyzes at least one of an intensity value or a wavelength value and generates the position value if the analyzed intensity value or the wavelength value is within one of a plurality of predefined ranges.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the invention. In the drawings:

FIG. 1-1 shows a perspective view of an endobronchial ultrasound (EBUS) bronchoscope capable of receiving a sheath device formed in accordance with an embodiment of the present disclosure;

FIG. 1-2 shows a close-up view of a distal end of the EBUS bronchoscope of FIG. 1-1;

FIG. 2 is an x-ray view of a distal end of a sheath formed in accordance with an embodiment;

FIG. 3 is a perspective view of the distal end of a scope with the sheath of FIG. 2 in a first position;

FIG. 4 is a perspective view of the distal end of the scope with the sheath of FIG. 2 in a second position;

FIG. 5 is a flow diagram of an exemplary process for forming at least a portion of the sheath of FIG. 2;

FIG. 6 is a block diagram of components of an imaging system formed in accordance with an embodiment of the present invention; and

FIG. 7 shows a flow diagram of an exemplary process performed by the imaging system of FIG. 6.

### DETAILED DESCRIPTION

The following description is merely exemplary in nature and is not intended to limit the present disclosure, application, or uses. The invention improves the way HCPs confirm that a sheath has exited a working channel of a scope and provides a visual indication that it is safe to deploy a medical instrument without fear of damaging the scope or surrounding tissue during a procedure.

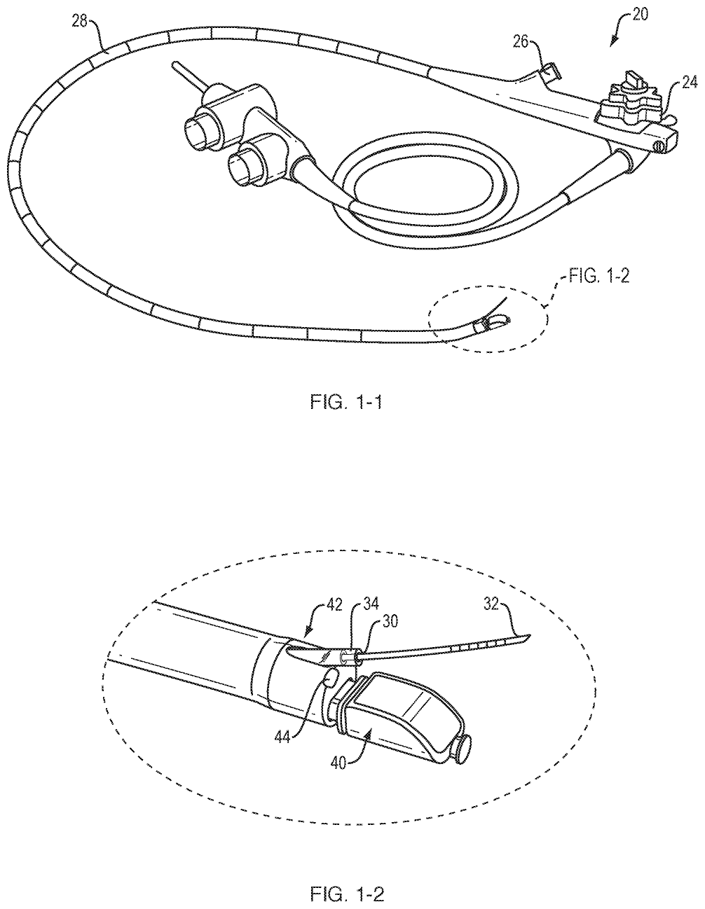

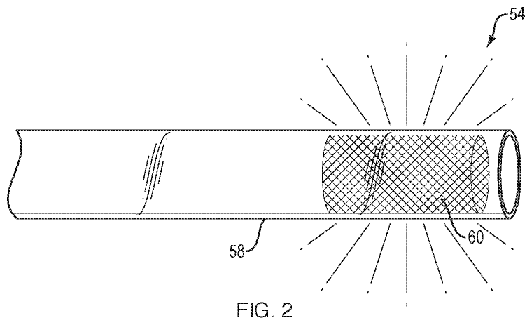

In one embodiment, FIGS. 1-1 and 1-2 shows an exemplary endoscope (e.g., an endobronchial ultrasound (EBUS) scope 20, such as an Olympus bronchoscope model # UC-180F), that is capable of receiving a medical device 30. The EBUS scope 20 includes a handle 24 with an input port 26 for receiving the medical device 30 and an insertion tube 28 connected to the handle 24. A distal end of the insertion tube 28 includes an ultrasound transducer head 40, an exit port 42 for an internal lumen (i.e., working channel) that connects to the input port 26, a camera 44 and a light. The medical device 30 passes through the working channel to extend at least partially out of the exit port 42.

In one embodiment, the medical device 30 includes a needle 32 received within a sheath 34. The sheath 34 includes a feature that is located at least at or near its distal end that will provide feedback to an operator as the distal end nears and exits the exit port 42. This feature provides a visual indication of where the distal end of the sheath 34 is located relative to the distal end of the working channel within the EBUS scope 20. In one embodiment, the sheath 34 includes a self-illuminating material that illuminates light and as the operator is feeding the sheath 34 through the bronchoscope, the operator will see more illumination or an indicator in an image produced by the camera 44 due to the sheath 34 produced light.

In one embodiment, a luminescent pigment powder (i.e., photoluminescence) is added to the material (e.g., resin) used to make at least a distal section of a sheath. The result is a glow in the dark sheath. As the sheath exits the working channel of the bronchoscope, the sheath radiates light that can be viewed in the image created by the camera (i.e., imaging system). This provides a visual cue of how far the sheath has extended from the bronchoscope. This visual clue allows the sheath operator to more easily understand sheath position and prevent the sheath from potentially damaging the airway walls.

The sheath could be made from polytetrafluoroethene (PTFE) and/or other materials. The sheath can be monolithic (single material) or composite (e.g. several layers, one of which could be a fiber braid or reinforcement material.

As shown in FIG. 2, an exemplary sheath 54 includes a laser cut hypo tube 60 that is received at a distal end of a flexible tube 58. The flexible tube 58 is composed of a biocompatible, flexible, luminescent material.

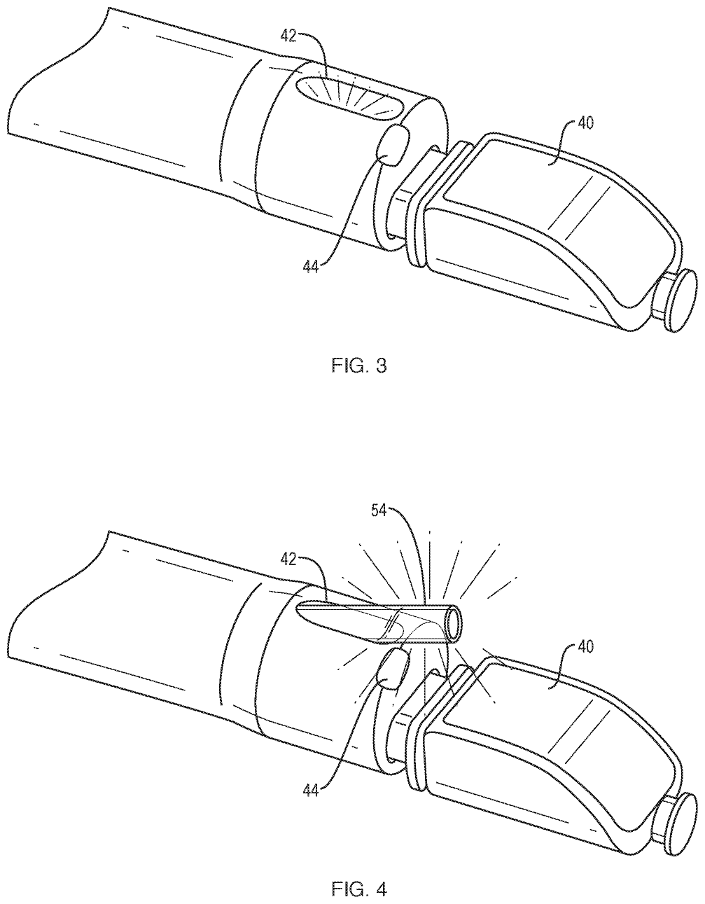

FIG. 3 illustrates a perspective view of the distal end of the EBUS bronchoscope 20 with the sheath 54 (not shown) located within a working channel that connects with the exit port 42. In this example, the sheath 54 is located near the end of the working channel. At this location the sheath 54 illuminates the walls of the exit port 42 but not much more as shown by the x's in FIG. 3.

FIG. 4 illustrates shows a similar view of the distal end of the EBUS bronchoscope 20 as in FIG. 3 with the sheath 54 protruding from the exit port 42. When the sheath 54, as shown, is located beyond the end of the working channel, it illuminates a greater amount of the housing and components at the distal end of the bronchoscope 20. As the sheath 54 protrudes further out of the exit port 42, the illumination becomes greater on surrounding components and surrounding tissue. Due to the proximity of the camera lens 44 to the exit port 42, the light produced by the sheath 54 will be received by the camera lens 44. The further the sheath 54 extends from the exit port 42 the greater the amount of light that will be communicated to an image processing device associated with the camera.

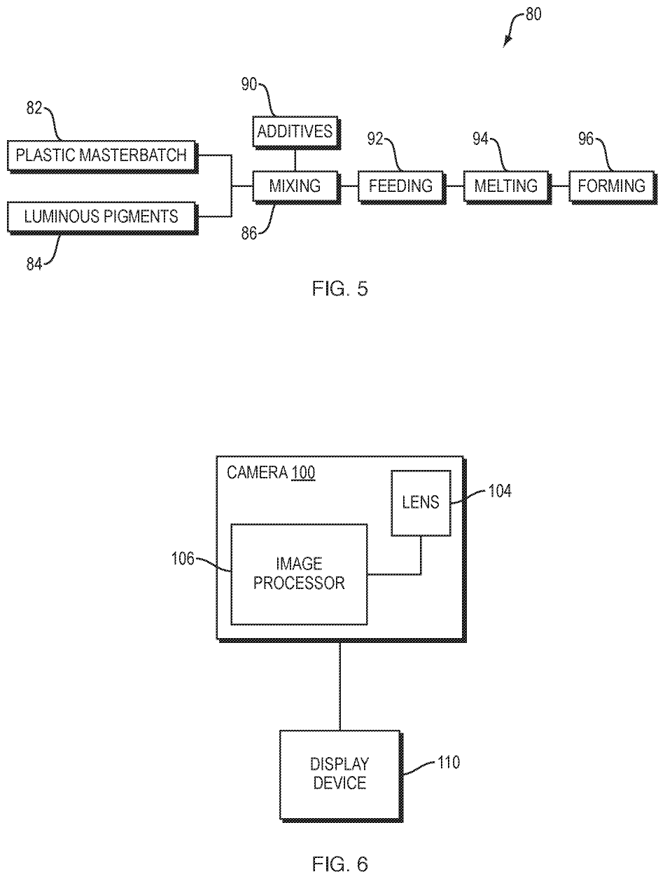

FIG. 5 illustrates an exemplary process 80 for creating a luminous sheath. First, a plastic masterbatch material 82 and a luminous pigment 84 are combined at a mixing step 86 with additives 90. The additives 90 include plasticizers, modifiers or other compounds that enhance the processability and/or mechanical properties of the desired tubing. The mechanical properties may include stiffness, resiliency, lubricity, etc.

Next, at a step 92, the mixed materials are moved to an extrusion press from a mixing chamber, for example by an extrusion ram. The ram pushes the mixed material into an extrusion die. In injection molding, for example, an auger moves plastic pellets into the press. The friction created in the augering process heats the plastic enough to melt. The temp may still be controlled through heaters.

Then, at a step 94, the mixed components are melted at a predefined temperature. The melted components are then formed into a sheath material by being forced through an extrusion die at step 96.

FIG. 6 illustrates an image processing system 100 included with components included within the EBUS scope 20 and components that electronically connect to the EBUS scope 20. The image processing system 100 includes a camera 102 and a display device 110. The camera 102 includes a lens or image sensor 104 and an image processor 106 that is in signal communication with the image sensor 104. The image sensor 104 is comparable to the camera lens 44 shown in FIG. 1-2. The image processor 106 is located remote from the EBUS scope 20.

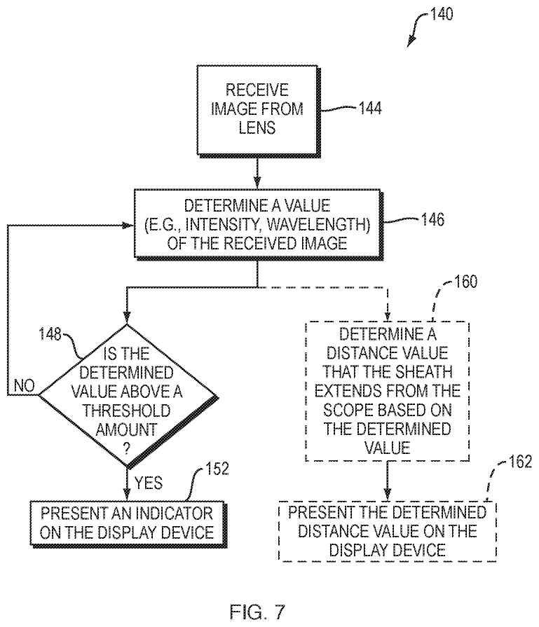

FIG. 7 illustrates an exemplary process 104 performed by the image processor 106 of FIG. 6. At a block 144 the image processor 106 receives an image from the image sensor 104. Next at a block 146, the image processor 106 determines a value associated with the received image. The image processor 106 may determine an intensity, a wavelength or some other value associated with the received image. At a decision block 148, the image processor 106 determines if the determined value is above a predefined threshold. If the determined value is not above the predefined threshold, the process returns to the block 144 to receive another image from the image sensor 104. If the determined value is above the predefined threshold, the image processor 106 produces an indicator that may be presented on the display device 110.

In an alternate embodiment, after the block 146, the image processor 106 determines at a block 160 a distance value associated with the determined value of the received image. Then, at a block 162, the image processor 106 presents the determined distance value on the display device 110. In one embodiment, the image processor 106 has been preprogrammed to include associations between light intensity values sensed within the received image and a position value of the sheath 54 relative to the exit port 42 of the EBUS scope 20. Light intensity values have been correlated to predefined sheath extension values that have been previously identified through comparisons of light amount values at different positions of the sheath 54 relative to the exit port 42

The features presented in the display device 110 as described above at blocks 152 and 162 may also be outputted in various other ways, such as an audible indicator outputted using an audio device.

Alternatively, the distal tip of the sheath could include a chamber with two suspended or separated liquids. Before inserting the sheath into the working channel of the bronchoscope the HCP would perform an action to mix the two liquids (i.e., like activating a glow stick) then insert the sheath into the bronchoscope. The distal tip would also provide a visual cue that the sheath has exited the working channel.

### Embodiments

A. A flexible sheath comprising: a distal end; a proximal end; and a lumen extending from the proximal end to the distal end, wherein the flexible sheath comprises a self-luminating feature, wherein the flexible sheath is configured to be received by a working channel of an endoscope.

B. The flexible sheath of A, wherein the endoscope is a bronchoscope.

C. The flexible sheath of A or B, wherein the self-luminating feature comprises a luminous pigment.

D. The flexible sheath of C, wherein the luminous pigment is applied as a paint to at least a portion of the flexible sheath.

E. The flexible sheath of C or D, wherein the luminous pigment is added during manufacturing of the flexible sheath.

F. The flexible sheath of any of A-E, further comprising: a first chamber section; a second chamber section; and a breakable membrane located between the first and second chamber sections, wherein the self-luminating feature comprises: at least a first solution located in the first chamber section; and at least a second solution located in the second chamber section, wherein when the first solution comes into contact with the second solution, a chemiluminescence activity occurs.

G. A system comprising: an endoscope system comprising: a handle; an insertion tube comprising: a proximal end coupled to the handle; a distal end, the proximal end being connected to the handle; a working channel; and an exit port of the working channel at the distal end of the insertion tube; and an imaging system comprising: an ultrasound transducer located at the distal end of the insertion tube; an imaging sensor configured to produce image data, the imaging sensor comprising a component located proximal of the ultrasound transducer and distal of the exit port at the distal end of the insertion tube; an image processor configured to receive the produced image data from the image sensor and generate an output signal based on the received image data; and an output device configured to output the generated output signal; and a flexible sheath configured to be received within the working channel, the flexible sheath comprising: a distal end; a proximal end; and a lumen extending from the proximal end to the distal end, wherein the flexible sheath includes a self-luminating feature.

H. The system of G, wherein the endoscope is a bronchoscope.

I. The system of G or H, wherein the self-luminating feature comprises a luminous pigment.

J. The system of I, wherein the luminous pigment is applied as a paint to at least a portion of the flexible sheath.

K. The system of any of G-J, wherein the luminous pigment is added during manufacturing of the flexible sheath.

L. The system of any of G-K, wherein the flexible sheath further comprises: a first chamber section; a second chamber section; and a breakable membrane located between the first and second chamber sections, wherein the self-luminating feature comprises: at least a first solution located in the first chamber section; and at least a second solution located in the second chamber section, wherein when the first solution comes into contact with the second solution, a chemiluminescence action occurs.

M. The system of any of G-L, wherein the image processor is configured to analyze the image data received from the image sensor and generate an indication about the distal end of the flexible sheath based on the analyzed image data, wherein the output device outputs the generated indication.

N. The system of M, wherein the image processor analyzes at least one of an intensity value or a wavelength value and generates the indication if the analyzed intensity value or the wavelength value is greater than a predefined threshold.

O. The system of any of G-N, wherein the image processor is configured to analyze the image data received from the image sensor and generate a value of a position of the distal end of the flexible sheath relative to the exit port based on the analyzed image data, wherein the output device outputs the generated value.

P. The system of O, wherein the image processor analyzes at least one of an intensity value or a wavelength value and generates the position value if the analyzed intensity value or the wavelength value is within one of a plurality of predefined ranges.

Q. The system of any of G-P, wherein the output device includes at least one of an audio or a visual output device.

The description of the invention is merely exemplary in nature and variations that do not depart from the gist of the invention are intended to be within the scope of the invention. Such variations are not to be regarded as a departure from the spirit and scope of the invention.

What is claimed is:

1. A flexible sheath comprising:
a distal end;
a proximal end; and
a lumen extending from the proximal end to the distal end,
wherein the flexible sheath comprises a self-luminating feature,
wherein the flexible sheath is configured to be received by a working channel of an endoscope.

2. The flexible sheath of claim 1, wherein the endoscope is a bronchoscope.

3. The flexible sheath of claim 1, wherein the self-luminating feature comprises a luminous pigment.

4. The flexible sheath of claim 3, wherein the luminous pigment is applied as a paint to at least a portion of the flexible sheath.

5. The flexible sheath of claim 3, wherein the luminous pigment is added during manufacturing of the flexible sheath.

6. The flexible sheath of claim 1, further comprising:
a first chamber section;
a second chamber section; and
a breakable membrane located between the first and second chamber sections,
wherein the self-luminating feature comprises:
at least a first solution located in the first chamber section; and
at least a second solution located in the second chamber section,
wherein when the first solution comes into contact with the second solution, a chemiluminescence activity occurs.

7. A system comprising:
an endoscope system comprising:
a handle;
an insertion tube comprising:
a proximal end coupled to the handle;
a distal end, the proximal end being connected to the handle;
a working channel; and
an exit port of the working channel at the distal end of the insertion tube; and
an imaging system comprising:
an ultrasound transducer located at the distal end of the insertion tube;
an imaging sensor configured to produce image data, the imaging sensor comprising a component located proximal of the ultrasound transducer and distal of the exit port at the distal end of the insertion tube;
an image processor configured to receive the produced image data from the image sensor and generate an output signal based on the received image data; and
an output device configured to output the generated output signal; and
a flexible sheath configured to be slidably received within the working channel, the flexible sheath comprising:
a distal end;
a proximal end; and
a lumen extending from the proximal end to the distal end,
wherein the flexible sheath includes a self-luminating feature.

8. The system of claim 7, wherein the endoscope is a bronchoscope.

9. The system of claim 7, wherein the self-luminating feature comprises a luminous pigment.

10. The system of claim 9, wherein the luminous pigment is applied as a paint to at least a portion of the flexible sheath.

11. The system of claim 9, wherein the luminous pigment is added during manufacturing of the flexible sheath.

12. The system of claim 7, wherein the flexible sheath further comprises:
a first chamber section;
a second chamber section; and
a breakable membrane located between the first and second chamber sections,
wherein the self-luminating feature comprises:
at least a first solution located in the first chamber section; and
at least a second solution located in the second chamber section,
wherein when the first solution comes into contact with the second solution, a chemiluminescence action occurs.

13. The system of claim 7, wherein the image processor generates the output signal by analyzing the image data received from the image sensor and generating an indication about the distal end of the flexible sheath based on the analyzed image data,
wherein the output device outputs the generated indication.

14. The system of claim 13, wherein the image processor analyzes at least one of an intensity value or a wavelength value of the image data and generates the indication if the analyzed intensity value or the wavelength value is greater than a predefined threshold.

15. The system of claim 7, wherein the image processor generates the output signal by analyzing the image data received from the image sensor and generating a value of a position of the distal end of the flexible sheath relative to the exit port based on the analyzed image data,
wherein the output device outputs the generated value.

16. The system of claim 15, wherein the image processor analyzes at least one of an intensity value or a wavelength value of the image data and generates the position value if the analyzed intensity value or the wavelength value is within one of a plurality of predefined ranges.

17. The system of claim 7, wherein the output device comprises at least one of an audio output device or a visual output device.
